(19) United States
(12) Patent Application Publication
(10) Pub. No.: US 2009/0142285 A1
(43) Pub. Date: Jun. 4, 2009

(54) COSMETIC COMPOSITION COMPRISING ASCORBIC ACID 2-GLUCOSIDE AND ERGOTHIONEINE

(75) Inventors: Robin KURFURST, Olivet (FR); Mathilde BONNET-DUQUENNOY, Vic Le Comte (FR)

Correspondence Address: FOLEY AND LARDNER LLP;SUITE 500, 3000 K STREET NW, WASHINGTON, DC, 20007, US

(73) Assignee: LVMH RECHERCHE

(21) Appl. No.: 12/275,151

(22) Filed: Nov. 20, 2008

(30) Foreign Application Priority Data
Nov. 30, 2007 (FR) 0759469

(51) Int. Cl. A61K 8/60; A61Q 19/02; A61Q 19/08

(52) U.S. Cl. 424/62; 514/25

(57) ABSTRACT

This invention relates to a cosmetic composition allowing the effects of skin aging to be reduced or delayed, comprising as the cosmetically active ingredients ascorbic acid 2-glucoside and ergothioneine. The invention also relates to the use of these cosmetically active ingredients in a cosmetic composition as agents to stimulate DNA repair enzymes, particularly enzymes to repair oxidised DNA bases in dermal and epidermal cells. Finally, the invention relates to a cosmetic treatment process involving this use.

The present invention relates to a cosmetic composition allowing the effects of skin aging to be reduced or delayed, comprising, as the cosmetically active ingredients, ascorbic acid 2-glucoside and ergothioneine. The invention also relates to the use of these cosmetically active ingredients in a cosmetic composition as agents to stimulate DNA repair enzymes, particularly enzymes to repair oxidised DNA bases in dermal and epidermal cells. Finally, the invention relates to a cosmetic treatment process involving this use.

Skin aging is determined by genetic and environmental factors.

More particularly, there are two types of skin aging:
Intrinsic or chronological aging which affects the skin in the same way as other organs and this corresponds to the inevitable changes resulting from the aging process,
Extrinsic aging related to environmental factors. The term <<actinic aging or heliodermatosis >> corresponds to clinical, histological and functional changes characteristic of skin exposed to the sun in a chronic manner and located in areas exposed to the sun.

These two processes are closely linked and in both cases the production of reactive oxygen species (ROS) results in oxidative stress and this determines the extent of skin aging.

Oxidative stress, also indifferently referred to in the literature as “oxidating stress”, is defined as an excess of free radicals present in the body resulting from excessive production by various physiological mechanisms or exogenous toxic phenomena.

Reactive oxygenated species (ROS) and free radicals can thus be produced either by cell metabolism such as mitochondrial respiration or by pathogenic infections, xenobiotic detoxification or sun radiation.

This physiological production of free radicals is controlled by cell defence systems and the balance of antioxidant/pro-oxidant defences is in equilibrium.

Whether it is as a result of a deficiency in antioxidants or antioxidant enzyme activity or overproduction of reactive oxygen species, a disequilibrium in the antioxidant/pro-oxidant defence balance leads to an excess of reactive oxygen species and free radicals which trigger a state called “oxidative stress” leading to oxidation of cell components including DNA (Sauvaigo S. et al, Brit. J. Dermatol., 157, 26-32, 2007).

Oxidative damage to DNA is extremely harmful for the cells and it has been shown that the half-a-million year survival of bacteria of the Arthrobacter species is linked to their ability to repair DNA (Stewart Johnson et al.; Proc. Natl. Acad. Sci., 104, 11401-14405, 2007).

In humans, in spite of cell defence systems, continual damage to DNA gradually alters the genome sequences and leads to the occurrence of cellular disorders such as aging (King et al., Mut. Res. 1994, 316, 79-90).

This suggests that physical or chemical attacks which increase the degree of DNA damage accelerate aging and consequently reduce life expectancy (Weirich-Schwaiger H. et al., 1994, Mut. Res., 316, 37-48).

Cosmetological research always strives to find effective ways to preserve the skin's essential functions in response to the passage of time and to attacks such as oxidative stress.

One first known means consists in preventing damage to cellular DNA, particularly to the DNA of cutaneous cells such as the keratinocytes, melanocytes or fibroblasts using compounds which act by chemically inhibiting reactive oxygen species before they are able to affect DNA.

Antioxidant agents are commonly used in cosmetic compositions for their ability to reduce or prevent damage caused by oxidative stress to skin cells.

From a chemical point of view, an antioxidant is a compound which acts as a reducing agent and therefore reacts with the oxidative species to deactivate and neutralise it and thus reduce or prevent oxidation of other chemical substances and stop chain reactions.

Antioxidants therefore prevent damage caused to the cells and cellular DNA by reactive oxygen species, free radicals and oxidised species formed under the effect of oxidative stress but are not chemically able to carry out subsequent repair of damage to cellular DNA.

Damage linked to oxidative stress is of two types. On the one hand, modification of the bases by a chemical oxidation process which leads to an alteration in the double helix sequence of DNA and, on the other hand, cuts in the single strand and double strand of cellular DNA.

DNA cellular damage is linked to the occurrence of diseases such as skin tumours.

Studies have also suggested that damage to DNA and/or repair of this damage is an important signal for the activation of melanin synthesis triggered by UV rays (Eller et al., 1996 Proc. Natl. Acad. Sci., 93 1087-92).

The study of this damage and of the repair mechanisms involved is also a major challenge in the fight against aging.

In fact, damage to cellular DNA through a cumulative process and as a result of its mutagenic capacity leads to cell damage and dysfunction which, in the skin, has the effect of promoting and activating cell aging.

The inventors have shown that in response to oxidative stress, the DNA repair capacity of fibroblasts from Japanese women decreases with age and that this decrease is aggravated by smoking (Sauvaigo et al., Int. J. Cosmet. Sci., 2005, 27, 76-79).

Many studies have focused on changes in cellular DNA resulting from oxidation of bases subjected to oxidative stress.

Among the many bases modified and reported in the literature, 8-Oxo-7,8-dihydro-2′-deoxyguanosine (8-oxo-dG) is a known biomarker of DNA lesions.

Studies conducted on human keratinocytes in vivo (Kang et al., Exp. Cell Res., 2005, 310, 186-195) reveal that 8-oxo-dG and reactive oxygen species accumulate in the DNA of senescent cells. These studies also show that the enzyme 8-oxo-dG DNA glycosylase (hOGG1), involved in the excision of 8-oxo-dG, is found in lower amounts in the same cells.

Authors have shown that in human skin fibroblast cultures, the accumulation of 8-oxo-dG in the DNA of aged cells is linked to a reduction in the activity of damage repair enzymes (Kaneko et al., Mutat Res., 2001, 487, 19-30).

Similarly, a study conducted on colorectal biopsy samples suggests an increase in 8-oxo-dG linked to the age of patients from whom the biopsies were taken (Tsurudome et al., Gerontol A Biol Sci Med Sci., 2001, 56, B483-5).

The elimination of oxidised bases from DNA involves an enzyme repair pathway by excision of oxidised bases, a pathway which is also found in many other living organisms (Boiteux et al., Biochimie, 1999, 81, 59-67).

The degree of expression of genes coding for the enzymes responsible for excision of oxidised bases from DNA is also a sensitive marker of oxidative stress (Powell et al. Cancer Letters, 2005, 22, 1-11).

A recent publication suggests that one means of reinforcing protection of DNA against UV radiation might be to administer cellular DNA repair enzymes in order to reduce sun-induced damage (Yaar and Gilchrest, Br J Dermatol. 2007 November; 157(5):874-87).

This invention is therefore based on investigation of a new pathway, other than exogenous administration of cellular DNA repair enzymes, which consists in direct stimulation of the activity of cellular DNA repair enzymes.

At present, there are very few methods which allow direct measurement of enzyme activity linked to the repair of altered DNA bases in the cells, especially as there are a large number of alterations thus leading to the need for a high degree of specificity.

The inventors of this patent application have studied the enzyme activity of repairing cellular DNA oxidised bases as a result of oxidative stress using a method which allowed them to specifically measure the activity of DNA repair enzymes for a set of modified bases characteristically produced as a result of oxidative stress.

In the course of this research, and against all odds, the Applicant found that ascorbic acid 2-glucoside and ergothioneine stimulate the repair enzyme activity of cellular DNA bases chemically altered by oxidative stress.

Stimulation of the activity of repair enzymes of the oxidised bases of cellular DNA improves the capacity for cellular DNA repair (a capacity which decreases with age) and more particularly prevention of the accumulation of oxidised bases in the cell, a source of cellular dysfunction and cutaneous aging.

It is thus possible to effectively combat cellular aging by using an active ingredient which stimulates cellular DNA repair enzyme activity and thus ensure that the intactness of cellular DNA is preserved in spite of damage caused by oxidative stress.

The Applicant also surprisingly and unexpectedly found that the repair enzyme activity of DNA is not uniformly stimulated by each of the compounds that were studied.

Each compound taken individually stimulates certain types of repair specifically over other types.

It is thus possible, by combining ascorbic acid 2-glucoside and ergothioneine, to cover a broader repair spectrum among the various known alterations to the double helix bases rather than if each molecule were used alone.

Finally, the Applicant has found that skin cells from a Caucasian donor and skin cells from an Asian donor do not respond in an identical manner to treatment by the same molecule when various oxidised base repairs were studied.

The combination of ascorbic acid 2-glucoside and ergothioneine in the same cosmetic composition allows a broader repair spectrum by the cellular enzymes of altered DNA to be covered, both for the Caucasian donor type and Asian donor type.

According to a first aspect, the invention therefore covers a cosmetic composition containing, in terms of cosmetically active ingredients, ascorbic acid 2-glucoside and ergothioneine, preferably L-ergothioneine, and at least one cosmetically acceptable excipient.

Ergothioneine is an established natural antioxidant effective against oxidative cell damage which more particularly inhibits cell death triggered by hydrogen peroxide and DNA oxidation caused by peroxinitrites in human cell lines (Aruoma et al., Food Chem Toxicol. 1999 November; 37(11):1043-53).

Ascorbic acid 2-glucoside, also called 2-O-α-D-Glucopyranosyl L-ascorbic acid, is an effective agent in protecting keratinocytes against cytotoxicity caused by UVB (Yasuda et al., In Vitro Cell Dev. Biol. Animal., 2004, 40, 71-73). Moreover, ascorbic acid 2-glucoside plays a role in protecting mouse skin cells placed in culture against damage caused by UVA or UVB, seen by a reduction in the cell DNA fragmentation (Masatsuji-Kato E et al., J. of Health Science, 2005, 51, 122-129).

According to a particular characteristic of this invention, ascorbic acid 2-glucoside is present in the cosmetic composition in an amount ranging from 0.0001% to 10%, advantageously from 0.001% to 5%, even more advantageously from 0.001% to 2.5%, and most preferably from 0.1% to 2.2% by weight with respect to the total weight of the composition.

According to another particular characteristic of the invention, ergothioneine is present in the cosmetic composition in a quantity ranging from 0.0001% to 10%, advantageously from 0.001% to 1%, even more advantageously from 0.005% to 0.8%, and most preferably from 0.01% to 0.5% by weight with respect to the total weight of the composition.

Advantageously, according to this invention, ergothioneine and ascorbic acid 2-glucoside are present in said cosmetic composition in an ergothioneine/ascorbic acid 2-glucoside ratio between 0.001 and 100, preferably between 0.002 and 80, even more preferably between 0.003 and 70, and most preferably of all between 0.005 and 60.

The composition according to the invention can also contain adjuvants commonly used in the field of cosmetics, such as stabilisers, antioxidants, solvents, fragrances, chelating agents, chemical or mineral water absorbers, mineral pigments, surfactants, polymers, silicon oils and dyes, this list not being limiting.

In a particular embodiment of this invention, the cosmetic composition also contains a depigmenting agent and/or agent intended to reduce the effects of cutaneous aging or to delay appearance of said effects.

The depigmenting agents known to the man skilled in the art are for example arbutin, kojic acid, azelaic acid, vitamin B3 or PP, calcium D-pantetheine-S-sulphonate, resorcinol derivatives, resveratrol, liquorice or white mulberry extracts, alpha-lipoic acid, linoleic acid, cation chelaters such as EDTA (ethylene diamine tetra acetic acid), soya extract, citrus unshui extract, diacetylboldine, this list being non-limiting. Preferably, the depigmenting agent is chosen from among the group comprised of vitamin B3, liquorice extract, calcium D-pantetheine-S-sulphonate, ferulic acid and its derivatives, citrus unshui extract and diacetylboldine.

Agents intended to reduce the effects of cutaneous aging or delay the appearance of said effects, known to the man skilled in the art, are advantageously chosen from among the group comprised of retinol, a retinol ester such as retinol propionate or retinol palmitate, beta-ecdysone, tocopherol derivatives such as tocopherol phosphate or potassium asorbyl tocopherol phosphate and asiaticoside.

The cosmetic composition according to the invention contains a cosmetically acceptable excipient, in other words an excipient compatible with the skin. It can advantageously be in any of the forms normally used for topical application, notably in the form of an aqueous, hydro-alcohol or oily solution, oil-in-water or water-in-oil emulsion, aqueous or oily gel, anhydrous liquid product, paste or solid or any other form for topical application.

This composition can be more or less fluid and has the appearance of a white or coloured cream, ointment, lotion, milk, serum, paste, foam or gel. It can be applied to the skin in the form of an aerosol or even a patch or mask or applicator. It can also be in a solid form, for example a free or compacted powder or stick. Advantageously the cosmetic composition is in the form of a lotion, gel, serum, cream, patch or mask.

Preferably, the cosmetic composition according to the invention is an emulsion in the form of a serum, day cream, night cream or after-sun cream.

In a particular embodiment of this invention, the cosmetic composition in one of the different forms is applied to at least one part of the body, particularly the hands or face.

Preferably, the cosmetic composition according to the invention in one of its different forms is applied after single or repeated exposure of the skin to oxidative stress or sunlight, particularly ultraviolet (UV) solar radiation.

According to a second aspect, the invention relates to the use of ascorbic acid 2-glucoside, in combination or not with ergothioneine, as a cosmetic active ingredient in a cosmetic composition intended to reduce the effects of skin aging or to delay the appearance of said effects.

The cosmetic composition according to the invention is more particularly aimed at reducing the effects of chronological and/or actinic aging of the skin or to delay the appearance of said effects.

Advantageously, the cosmetic composition makes it possible to reduce or delay the formation of wrinkles and/or to reduce or delay loss of skin firmness and/or to reduce or delay pigment patches on the skin, particularly sun-induced pigment patches and/or age spots. The cosmetic composition is advantageously that which is defined above.

According to another preferred embodiment of the invention, the cosmetic composition is intended to stimulate enzymes which repair damage caused to DNA by single or repeated exposure of the skin to oxidative stress or sunlight, particularly ultraviolet radiation (UV).

The cosmetic composition is more particularly intended to stimulate the enzymes which repair the oxidised bases of dermal and epidermal cellular DNA.

In a particular embodiment, use according to this invention is characterised in that the cosmetic composition contains ascorbic acid 2-glucoside and ergothioneine. Advantageously, ergothioneine and ascorbic acid 2-glucoside are present in said cosmetic composition in an ergothioneine/ascorbic acid 2-glucoside ratio of 0.001 to 100, preferably from 0.002 to 80, even more preferably from 0.003 to 70, and most preferably still from 0.005 to 60.

The advantageous quantities of ascorbic acid 2-glucoside and ergothioneine are those indicated above.

Advantageously, the cosmetic composition also contains a cosmetically acceptable excipient and at least one depigmenting agent and/or cutaneous anti-aging agent. The preferred depigmenting agents and/or agents intended to reduce the effects of skin aging or to delay the appearance of said effects are those indicated above.

According to a final aspect, this invention relates to a process for cosmetic treatment aimed at reducing the effects of skin aging or delaying the appearance of said effects characterised in that a sufficient amount of the cosmetic composition containing ascorbic acid 2-glucoside and ergothioneine as the cosmetically active ingredients is applied to the skin. This composition is advantageously the one described above.

The cosmetic treatment process is aimed more particularly at reducing the effects of chronological and/or actinic aging affecting the skin or delaying the appearance of said effects.

Advantageously, the process according to the invention is characterised in that the cosmetic composition makes it possible to reduce or delay the formation of wrinkles and/or reduce or delay the loss of skin firmness and/or reduce or delay skin pigment patches, particularly sun-induced pigment spots and/or age spots.

Preferably, the process according to the invention is characterised in that the cosmetic composition in one of its different forms is applied to at least one part of the body, particularly the hands or face.

Equally preferably, the process according to the invention is characterised in that the cosmetic composition in one of its different forms is applied following single or repeated exposure of the skin to oxidative stress or sunlight, particularly ultraviolet sun radiation (UV).

The methods of administration, doses and optimum forms of the compositions according to the invention can be decided in accordance with the customary criteria used to produce cosmetic treatment products.

The figures and examples below are given purely for the purpose of illustration and are in no way limiting.

### FIGURES

FIG. 1: Principle of the measurement of DNA-N-glycosylase activity in the biochip.

FIG. 2: Lesions studied and distribution diagram on the biochip
Uracil=uracil
OxodG=8-oxo Gua=8-Oxo-7,8-dihydro-2′-desoxyguanosine
Inosine=inosine
HmdU=5 hydroxymethyl-2′-desoxyuridine
Formylamine=N-(desoxy-beta-D-erythropentofuranosyl)-formylamine
EthenodA=N6-etheno-desoxyadenosine

FIG. 3: Effect of ergothioneine on the digestion of control oligonucleotide (no lesion) after 24 hour treatment.

FIG. 4: Effect of ascorbic acid 2-glucoside on the digestion of control oligonucleotide (no lesion) after 24 hour treatment.

FIG. 5: Effect of ergothioneine on the digestion of various fibroblast lesions taken from a Caucasian donor immediately after treatment (A) or 24 hours after treatment (B).

The values reported are the residual percentages of normalised lesions with respect to the control oligonucleotide.

A value in excess of 100% indicates that the treatment inhibits lesion repair.

A value below 100% indicates that the treatment increases lesion repair.

### EXAMPLES

### Example 1

### Measurement of the Biological Activity of Treatment Respectively by Ergothioneine and Ascorbic Acid 2-Glucoside on the Capacity of Normal Human Fibroblasts in Culture to Repair DNA Damage

Fibroblasts were obtained from a breast sample from a 52-year-old Caucasian woman and from photo-protected skin of a 39-year-old Asian woman (Japanese).

1.1. Methods

### 1.1.1. Fibroblast Treatment

Fibroblasts from each of the donors, Caucasian (FHN52) and Asian (FHN 1C), were cultured and treated in the exponential growth phase.

Fibroblasts were treated with ergothioneine (AGI Dermatics, Newport, N.Y., USA) at a non-cytotoxic concentration of 4 mM for 24 hours.

Treatment of fibroblasts with ascorbic acid 2-glucoside (Siber Hegner, Miribel, France) was carried out at a non-cytotoxic concentration of 50 μM of ascorbic acid 2-acid for 24 hours.

Cells were then rinsed and isolated by trypsin treatment.

Non-treated controls (NT) were made up in parallel for each point.
Cell pellets were frozen in culture medium (Medium 199+Earle's+L-glutamine completed with SVF, penicillin and streptomycin)+10% DMSO. Six 100 mm Petri dishes were used for each condition.

### 1.1.2. Preparation of Extracts

Nuclear extracts were prepared in order to obtain protein solutions containing the repair enzymes of interest.

Proteins were assayed with a Micro BCA kit (Interchim, Montlucon, France).

Extracts were aliquoted per 5 μl fraction and were frozen at −80° C.

Extracts were used at a final concentration of 10 μg/ml.

After being defrosted twice, they were eliminated.

### 1.1.3. OLISA Technology Applied to DNA Repair

OLISA technology (Bonnet-Duquennoy et al., Eur. J. Dermatol., 16, 2, 136-140 2006) was used to measure the excision of nucleic bases modified by glycosylase type DNA repair enzymes.

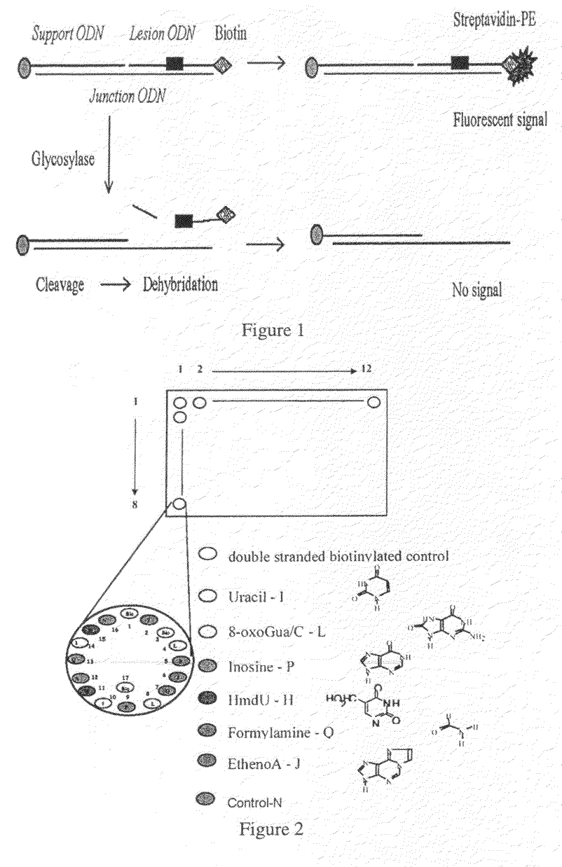

The principle (FIG. 1) is described in the publication entitled: “An oligonucleotide micro-array for the monitoring of repair enzyme activity toward different DNA damage”, Sauvaigo et al., Annal. Biochem. (2004); 333: 182-192.

Labelled oligonucleotide probes immobilised on the support generate synthetic double strand DNA containing in their base sequence, the altered base whose repair activity by cellular enzymes is being studied.

The probes were determined in order to study enzyme repair typical lesions resulting from exposure to oxidative stress (FIG. 2).

An OLISA format chip (96-well plate; bioMérieux, Grenoble, France) with 17 spots was functionalised by oligonucleotides containing the various lesions of interest (LAN synthesis, CEA, Grenoble, France).

For this study, 2 spots per lesion of interest or control oligonucleotide (without lesion) and 3 spots used to direct the readings were used.

Functionalised wells were incubated at 30° C. in the presence of the extract of interest.

The duration of digestion chosen is 45 minutes for the experiments. Extracts were deposited in four wells.

In parallel, extract-free control wells (buffer only) were also made up.

After reading the chips for each condition (or slide), 8 values per lesion of interest were obtained (2 spots×4 wells)

The mean of these values was calculated for “control” wells as well as for “extract” wells, with lesions remaining undigested in the “control” wells.

Thus for each lesion, the percentage of lesions remaining after the extract activity is determined by calculating the ratio of “extract” well signals with respect to control wells which represent 100% lesions (calculation carried out using the mean of the 8 values obtained).

8 percentage values were obtained for each residual lesion.

The signal corresponds to the quantity of remaining undigested probe as well as a low signal corresponding to high digestion and therefore to high enzyme activity.

Each experiment was carried out in duplicate or triplicate.

### 1.1.4. Statistical Analyses

In order to determine the effect of ergothioneine, on the one hand, and of ascorbic acid 2-glucoside, on the other, on non-specific digestion of extracts, data obtained with the control oligonucleotide normalised by the control were used.

Similarly, to determine the effect of each of the 2 active ingredients on specific digestion of extracts, data obtained for each of the lesions were normalised by data obtained with the control oligonucleotide normalised by the control.

Experiments were analysed independently in order to take into account variability in the manufacture of chips.

In the same way, an analysis was carried out for each of the 2 donors tested. Only the activity on the control oligonucleotide was carried out for the 2 donors.

The statistical test used was ANOVA variant analysis.

1.2. Results:

### 1.2.1 Activity of Ergothioneine on Non-Specific Digestion Activity

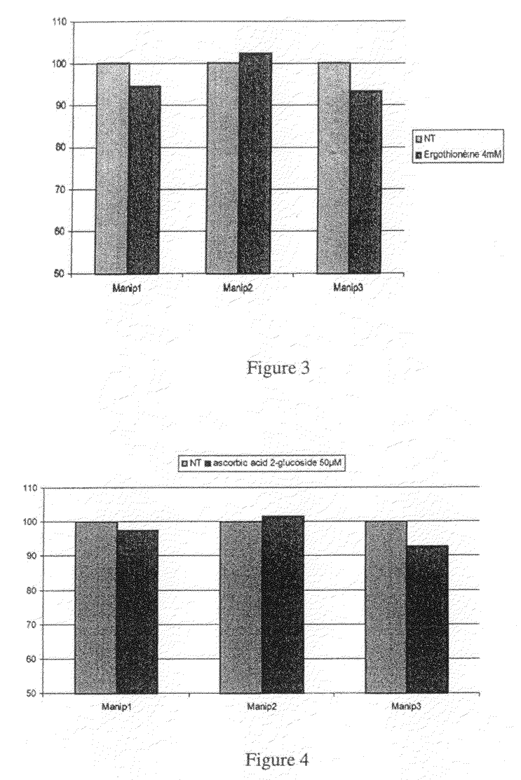

Treatment by ergothioneine for 24 hours had no significant effect on the control oligonucleotide immediately after treatment (refer to FIG. 3).

This indicates that treatment does not modify the non-specific digestive activity (nuclease) of cell extracts.

### 1.2.2 Effect of Ascorbic Acid 2-Glucoside on Non-Specific Digestion Activity:

Incubation of ascorbic acid 2-glucoside for 24 hours did not have a significant effect on the control oligonucleotide immediately after treatment (refer to FIG. 4).

This indicates that treatment does not modify the non-specific digestive activity (nuclease) of cell extracts.

### 5.2.3 Activity of Ergothioneine on Specific Digestive Activity:

TABLE 1
Summary of results of treatment with
ergothioneine of 2 donors 24 hours after treatment:

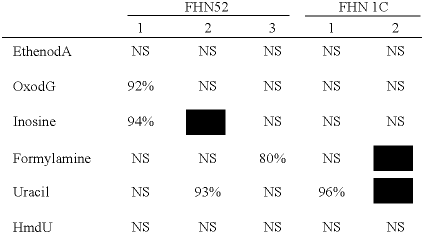

The reported values are the residual percentages for normalised lesions with respect to the control oligonucleotide.

A value in excess of 100% indicates that the treatment inhibits lesion repair.

A value below 100% indicates that the treatment increases lesion repair.

In bold 0.05<p<0.15. Shaded 0.05<p. N.S: non-significant variation

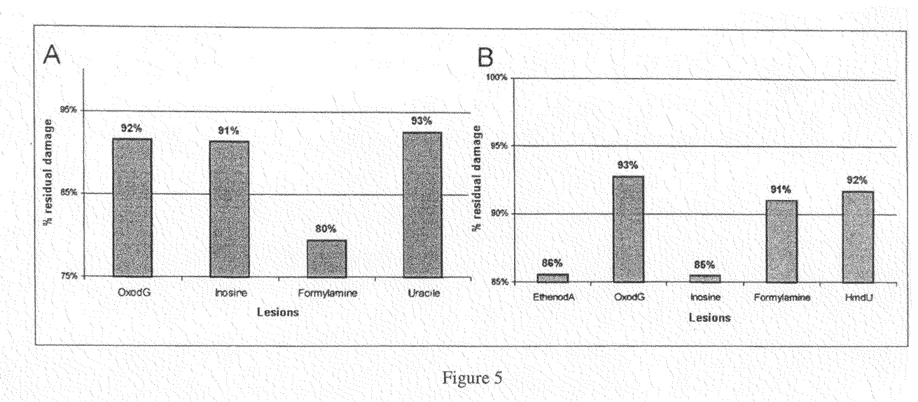

It can be seen from the table that:
The Caucasian donor (FHN 52) is more responsive than the Asian donor (FHN 1C).
The residual percentage for the 4 lesions: 8 oxodG, inosine, formylamine and uracil decreases significantly immediately after treatment with ergothioneine in the Caucasian donor (FHN 52) (refer to FIG. 5)
The residual percentage of formylamine and uracil lesions decreases significantly immediately after treatment with ergothioneine in the Asian donor.

An additional study was carried out on Caucasian donor cells (FHN 52) in order to measure the long-term effects of ergothioneine on the ability of fibroblasts to repair DNA.

Cells were incubated for 24 hours with ergothioneine (4 mM), rinsed and recultured (without ergothioneine) for a further 24 hours.

FIG. 5 shows that the residual percentage of ethenoda, 8-oxo-dG, inosine, formylamine and HmdU lesions decreases significantly in cells treated for 24 hours then cultured for a further 24 hours.

### 1.2.4 Conclusions:

These results show that treatment with ergothioneine at a concentration of 4 mM significantly increases digestion activity and therefore elimination of modified DNA bases formed as a result of oxidative stress.

The increase in enzyme activity induced in fibroblast extracts demonstrates that ergothioneine stimulates DNA repair within normal human fibroblasts.

This stimulation remains measurable 24 hours after treatment with ergothioneine. This observation suggests long-term efficacy of this treatment on DNA repair.

### 1.2.5 Effect of Ascorbic Acid 2-Glucoside on Specific Digestive Activity.

TABLE 2
Summary of the results of treatment with
ascorbic acid 2-glucoside in 2 donors 24 hours after treatment:

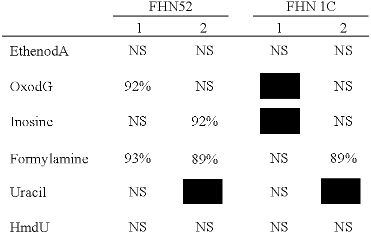

The values reported are the residual percentages of normalised lesions with respect to the control oligonucleotide.

A value in excess of 100% indicates that the treatment inhibits lesion repair.

A value below 100% indicates that the treatment increases lesion repair.

In bold 0.05<p<0.15. Shaded 0.05<p. N.S: non-significant variation

It is observed that:
The residual percentage of 4 lesions: 8-oxo-dG, inosine, formylamine and uracil decreases significantly 24 hours after treatment with ascorbic acid 2-glucoside in the Caucasian donor (FHN 52)
The residual percentage of 4 lesions: 8 oxodG, inosine, formylamine and uracil decreases significantly 24 hours after treatment with ascorbic acid 2-glucoside in the Asian donor (FHN 1C).

### 1.2.6 Conclusion

These results show that treatment with ascorbic acid 2-glucoside at a concentration of 50 μM significantly increases digestion activity and therefore elimination of modified DNA bases formed as a result of oxidative stress.

The increase in enzyme activity in fibroblast extracts shows that ascorbic acid 2-glucoside stimulates DNA repair within normal human fibroblasts obtained from a Caucasian or Asian donor.

This stimulation remains measurable 24 hours after treatment with ascorbic acid 2-glucoside.

This observation suggests long-term efficacy of this treatment with regard to DNA repair.

### Example 2

### Face Cream

The proportions of the following 4 compositions are given as the percent by weight with respect to total weight.

| | |
|---|---|
| Cyclopentasiloxane | 12.90 |
| Butylene Glycol | 11.00 |
| PEG-7 Dimethicone | 5.00 |
| Phenyl Trimethicone | 2.50 |
| Glycerin | 2.00 |
| Vinyl dimethicone/methicone | 1.50 |
| silsesquioxane crosspolymer | |
| Neopentyl glycol diethylhexanoate | 1.00 |
| Silica dimethyl silylate | 0.80 |
| Sodium Citrate | 0.50 |
| Sodium Chloride | 0.50 |
| Trimethylsiloxysilicate | 0.50 |
| Preservative | 0.70 |
| Tocopheryl acetate | 0.20 |
| Ergothioneine | 0.01 |
| Tetrasodium EDTA | 0.10 |
| Fragrance | 0.04 |
| Sodium Hyaluronate | 0.01 |
| Potassium Sorbate | <0.01 |
| Water | qsp 100% |

### Example 3

### Night Serum for the Eye Contour

| | |
|---|---|
| Glycerin | 3.05 |
| Butylene glycol | 3.00 |
| Squalane | 2.90 |
| Cetearyl ethylhexanoate | 2.60 |
| Ascorbic acid 2-glucoside | 2.00 |
| Steareth-21 | 1.20 |
| Myristyl myristate | 1.00 |
| Polymethyl methacrylate | 1.00 |
| Glyceryl stearate | 0.80 |
| Preservatives | 0.70 |
| Mica | 0.65 |
| Sodium Citrate | 0.45 |
| Ketyl alcohol | 0.40 |
| Stearyl alcohol | 0.40 |
| Sodium Hydroxide | 0.30 |
| Acrylates/C10-30 alkyl acrylate | 0.30 |
| crosspolymer | |
| Polyacrylamide | 0.24 |
| Xanthane gum | 0.20 |
| Dimethicone | 0.20 |
| Tetrasodium EDTA | 0.20 |
| C13-14 Isoparaffin | 0.15 |
| Stearic acid | 0.05 |
| Palmitic acid | 0.05 |
| Fragrance | 0.03 |
| Laureth-7 | 0.03 |
| Citric acid | 0.02 |
| Potassium sorbate | <0.01 |
| Water | qsp 100% |

### Example 4

### Essence

| | |
|---|---|
| Butylene glycol | 7.00 |
| Glycerin | 4.00 |
| Polymethylsilsesquioxane | 4.00 |
| Ascorbic acid 2-glucoside | 2.00 |
| Isohexadecane | 1.50 |
| Cyclopentasiloxane | 1.30 |
| Aminomethyl propanediol | 1.00 |
| Acrylates/C10-30 alkyl acrylate | 0.45 |
| crosspolymer | |
| Sodium citrate | 0.45 |
| Preservatives | 0.70 |
| Dimethicone | 0.20 |
| Tetrasodium EDTA | 0.20 |
| PEG-7 Glyceryl cocoate | 0.20 |
| Ergothioneine | 0.10 |
| Xanthane gum | 0.05 |
| Citric acid | 0.02 |
| Fragrances | 0.02 |
| Potassium sorbate | <0.01% |
| Water | qsp 100% |

### BIBLIOGRAPHIC REFERENCES

Sauvaigo S. et al, Brit. J. Dermatol., 157, 26-32, 2007
Stewart Johnson et al.; Proc. Natl. Acad. Sci., 104, 11401-14405, 2007
King et al., Mut. Res. 1994, 316, 79-90
Weirich-Schwaiger H, et al., 1994, Mut. Res., 316, 37-48
Eller et al., 1996 Proc. Natl. Acad. Sci., 93 1087-92
Sauvaigo et al., Int. J. Cosmet. Sci., 2005, 27, 76-79
Kang et al., Exp. Cell Res., 2005, 310, 186-195
Kaneko et al., Mutat Res., 2001, 487, 19-30
Tsurudome et al., Gerontol A Biol Sci Med Sci., 2001, 56, B483-5
Boiteux et al., Biochimie, 1999, 81, 59-67
Powell et al. Cancer Letters, 2005, 22, 1-11
Yaar and Gilchrest, Br J Dermatol. 2007 November; 157(5):874-87
Aruoma et al., Food Chem Toxicol. 1999 November; 37(11):1043-53
Yasuda et al., In Vitro Cell. Dev. Biol. Animal., 2004, 40, 71-73
Masatsuji-Kato E et al., J. of Health Science, 2005, 51, 122-129
Bonnet-Duquennoy et al., Eur. J. Dermatol., 16, 2, 136-140 2006
Sauvaigo et al., Annal. Biochem. (2004); 333: 182-192

1. Cosmetic composition comprising as the cosmetically active ingredients ascorbic acid 2-glucoside and ergothioneine and at least one cosmetically acceptable excipient.

2. Cosmetic composition according to claim 1 wherein the ergothioneine is L-ergothioneine.

3. Cosmetic composition according to claim 1 or 2 characterized in that the ascorbic acid 2-glucoside is present in an amount ranging from 0.0001% to 10%, advantageously from 0.001% to 5%, even more advantageously from 0.001% to 2.5%, and most preferably from 0.1% to 2.2% by weight with respect to the total weight of the composition.

4. Cosmetic composition according to one of the preceding claims characterized in that the ergothioneine is present in the cosmetic composition in a quantity ranging from 0.0001% to 10%, advantageously from 0.001% to 1%, even more advantageously from 0.005% to 0.8%, and most preferably from 0.01% to 0.5% by weight with respect to the total weight of the composition.

5. Cosmetic composition according to one of the preceding claims characterized in that the ergothioneine and ascorbic acid 2-glucoside are present in said cosmetic composition in an ergothioneine/ascorbic acid 2-glucoside ratio between 0.001 and 100, preferably between 0.002 and 80, even more preferably between 0.003 and 70, and most preferably of all between 0.005 and 60.

6. Cosmetic composition according to one of the preceding claims further comprising at least a depigmenting agent and/or an agent intended to reduce or delay the effects of cutaneous aging, like the effects of chronological and/or actinic aging.

7. Cosmetic composition according to one of the preceding claims characterized in that the depigmenting agent is chosen from among the group comprised of vitamin B3, calcium D-pantetheine-S-sulphonate, liquorice extract, ferulic acid and its derivatives, citrus unshiu extract and diacetylboldine.

8. Cosmetic composition according to one of the preceding claims characterized in that the agent intended to reduce the effects of cutaneous aging or to delay the appearance of said effects is chosen from the group comprised of retinol, a retinol ester such as retinol propionate or retinol palmitate, beta-ecdysone, tocopherol derivatives such as tocopherol phosphate or potassium asorbyl tocopherol phosphate and asiaticoside.

9. Cosmetic composition according to one of the preceding claims characterized in that it is in the form of a powder, possibly compacted, a lotion, a gel, a serum, a cream, a patch or a mask.

10. Cosmetic composition according to claim 9 characterized in that it is an emulsion in the form of a day cream, a night cream or an after-sun cream.

11. Use of ascorbic acid 2-glucoside, in combination or not with ergothioneine, as a cosmetic active ingredient in a cosmetic composition intended to reduce the effects of skin aging or to delay the appearance of said effects.

12. Use according to claim 11 characterized in that the cosmetic composition makes it possible to reduce or delay the formation of wrinkles and/or to reduce or delay loss of skin firmness and/or to reduce or delay pigment patches on the skin, particularly sun-induced pigment patches and/or age spots.

13. Use according to claim 11 or 12 characterized in that the cosmetic composition is intended to stimulate enzymes which repair damage caused to DNA by single or repeated exposure of the skin to oxidative stress or sunlight, particularly ultraviolet (UV) solar radiation.

14. Use according to claim 13 characterized in that the cosmetic composition is intended to stimulate the enzymes which repair the oxidised bases of dermal and epidermal cellular DNA.

15. Use according to any one of claims 11 to 14 characterized in that the ascorbic acid 2-glucoside is present in an amount ranging from 0.0001% to 10%, advantageously from 0.001% to 5%, even more advantageously from 0.001% to 5%, and most preferably from 0.1% to 2.2% by weight with respect to the total weight of the composition.

16. Use according to any one of claims 11 to 15 characterized in that the cosmetic composition contains ascorbic acid 2-glucoside and ergothioneine.

17. Use according to any one of claims 11 to 16 characterized in that ergothioneine is present in the cosmetic composition in a quantity ranging from 0.0001% to 10%, advantageously from 0.001% to 1%, even more advantageously from 0.005% to 0.8%, and most preferably from 0.01% to 0.5% by weight with respect to the total weight of the composition.

18. Use according to claim 16 or 17 characterized in that ergothioneine and ascorbic acid 2-glucoside are present in said cosmetic composition in an ergothioneine/ascorbic acid 2-glucoside ratio between 0.001 and 100, preferably between 0.002 and 80, even more preferably between 0.003 and 70, and most preferably of all between 0.005 and 60.

19. Use according to any one of claims 11 to 18 characterized in that the cosmetic composition also contains a cosmetically acceptable excipient and at least one depigmenting agent and/or agent intended to reduce the effects of skin aging or to delay the appearance of said effects.

20. Use according to claim 19 characterized in that the depigmenting agent is chosen from among the group comprised of vitamin B3, calcium D-pantetheine-S-sulphonate, liquorice extract, ferulic acid and its derivatives, citrus unshiu extract and diacetylboldine.

21. Use according to claim 19 or 20 characterized in that the agent intended to reduce the effects of cutaneous aging or to delay the appearance of said effects is chosen from among the group comprised of retinol, a retinol ester such as retinol propionate or retinol palmitate, beta-ecdysone, tocopherol derivatives such as tocopherol phosphate or potassium asorbyl tocopheryl phosphate and asiaticoside.

22. Use according to any one of claims 11 to 21 characterized in that the cosmetic composition is in the form of a powder, possibly compacted, a lotion, a gel, an emulsion, a patch or a mask.

23. Use according to claim 22 characterized in that cosmetic composition in one of the different forms is applied to at least one part of the body, particularly the hands or face.

24. Use according to claim 22 or 23 characterized in that the cosmetic composition in one of its different forms is applied after single or repeated exposure of the skin to oxidative stress or sunlight, particularly ultraviolet (UV) solar radiation.

25. Cosmetic treatment process to reduce or delay the effects of aging on the skin, such as the effects of chronological and/or actinic aging, characterized in that a cosmetic composition according to any one of claims 1 to 10 is applied to the skin in a sufficient quantity to reduce the effects of cutaneous aging or to delay the appearance of said effects.

26. Process according to claim 25 characterized in that the cosmetic composition makes it possible to reduce or delay the formation of wrinkles and/or to reduce or delay loss of skin firmness and/or to reduce or delay pigment patches on the skin, particularly sun-induced pigment patches and/or age spots.

27. Process according to claim 25 or 26 characterized in that the cosmetic composition in one of its different forms is applied to at least one part of the body, particularly the hands or face.

28. Process according to any one of claims 25 to 27 characterized in that the cosmetic composition is in the form of a lotion, a gel, an emulsion, a patch or a mask.

29. Process according to claim 28 characterized in that the cosmetic composition is an emulsion in the form of a day cream, night cream or after-sun cream.

30. Process according to claims 27 to 29 characterized in that the cosmetic composition in one of its different forms is applied after single or repeated exposure of the skin to oxidative stress or sunlight, particularly ultraviolet (UV) solar radiation.
